(19) United States
(12) Patent Application Publication
(10) Pub. No.: US 2024/0108671 A1
(43) Pub. Date: Apr. 4, 2024

(54) HERBAL MIXTURE FOR SMOKING IN A SHISHA DEVICE

(71) Applicants: Jonnell M. Miller, Windsor, CO (US); Adrian Garcia, Northglenn, CO (US)

(72) Inventors: Jonnell M. Miller, Windsor, CO (US); Adrian Garcia, Northglenn, CO (US)

(21) Appl. No.: 18/479,799

(22) Filed: Oct. 2, 2023

Related U.S. Application Data
(60) Provisional application No. 63/412,490, filed on Oct. 2, 2022.

(51) Int. Cl. A61K 36/185; A24B 15/167; A24B 15/30; A24F 1/30; A61K 31/00

(52) U.S. Cl. CPC A61K 36/185; A24B 15/167; A24B 15/303; A24F 1/30; A61K 31/658; A61K 2236/15; A61K 2236/51

(57) ABSTRACT

A smokable and edible material usable in a shisha device and method of making the same. The method includes providing fresh hemp leaf material. Shredding the hemp leaf material into pieces having a maximum length of ½-2″ and preferably 1-2″ in length. The width is preferably between 0.25 and 1″. Next the method rinses the shredded hemp leaf material to remove chlorophyll, water soluble impurities including heavy metals, pesticides and natural residues. This may include 3-6 washes of warm water (up to 212° F.). In a preferred embodiment there is no need for an ethanol bath to remove crystallized cannabinoids because the leaf material does not typically express crystallized cannabinoids. The step of drying follows immediately after rinsing. Drying includes pressing and fluffing the hemp leaf material then drying in a dehydrator.

### FIELD OF THE INVENTION

The present invention relates to ways of making smokable herbage, including cannabis. More particularly, the present invention relates to a way of making and presenting smokable hemp that is non-addictive and that has specific components that optimize flavor and effect.

### BACKGROUND AND SUMMARY OF THE INVENTION

Shisha is sometimes referred to as hooka, nargileh, kalian or water pipe. Shisha smoking evolved, according to one historical theory, at the time of the first introduction of tobacco in India around the year 1560 and it soon spread to Persia, Turkey, Egypt and other places. Over the generations and many cultural traditions, Shisha has become rooted as a social event where many share a tobacco with a shisha water pipe. Other historical theories indicate the invention of shisha devices in Iran prior to the year 1560. Currently, there is a renaissance in popularity of using shisha devices.

It is also recorded that tobacco in those days was quite harsh and perhaps laden with tar, carbon particles, and various harsh plant terpinoids that would irritate the lungs of smokers. Some believe that a man named Hakim About Futteh Gilani created an early version of a shisha device in India in order to yield a healthier way of smoking by bubbling the smoke through water. Shisha devices were mostly used by nobility, thus it was embedded in many minds as social class significator.

Some shisha devices have many hose ports so each smoker can use one. Others have a single hose port and smokers share the shisha in this way.

The standard components of a shisha devices include a bowl, head gasket, tray, stem, release valve, hose port and hose gasket, down stem, vase, and a hose with a smoking tip. The vase holds water to enable the down stem to deliver smoke from the bowl to bubble through the water, mellowing the flavor, heat and toxicity of the smoke. The result is a mild tobacco smoke that flows via the hose port via and the hose in response to a smoker drawing an inhalation from the smoking tip of the hose.

Tobacco was the primary herb smoked in shisha devices. Sometimes the tobacco is mixed with molasses and spices. A smoldering charcoal chunk is suspended above the tobacco when it is placed in a ceramic bowl of a shisha device for smoking. This charcoal heats the tobacco to enable to also smolder. The ceramic bowl holds heat to enable the tobacco to smolder for long periods and to enable smoking in a relaxed and unrushed manner.

Sadly, tobacco is quite addictive, and many in the modern era shun the use of addictive substances like tobacco. Even so, there is a social need for many to continue the cultural traditions of shisha, and expand them in western markets. What is desired is a way of having a shisha based social experience that is simple, not messy, non-addictive, and that maintains ignition for long periods (longer than a few minutes).

Presently, a product called “Ooka” that is marketed at www.getooka.com and is a shisha vaporizer that heats shisha made from marijuana and vaporizes the marijuana in hooka. The hooka has an electrical heating element to eliminate the need for charcoal as a combustible heating element. The shisha is packed into a pod for ease of use. The pod is generally cylindrical or a conical frustum shape, having two ends with a multitude of perforations to enable air flow through the pod. The pod is heated to vaporize the enclosed marijuana material. While the Ooka device is a step forward in the art of hookas and shisha consumption, not all customers desire the psychoactive effects of marijuana.

One drawback of the use of a pod is that airflow may be inhibited by the compressed vaporizable herbage contained therein. Shisha is relatively wet or sticky and often can limit airflow through the material, particularly when compressed in a pod.

### SUMMARY AND DETAILED DESCRIPTION OF THE INVENTION

The present invention includes herbage specifically designed for long lasting vaporization suitable for smoking in a shisha device.

For example, the herbage can be pressed into a pod. A typical pod has a cylindrical shape, having a uniform diameter and a height. In a preferred embodiment the pod has a conical frustum shape. Both the conical frustum and cylindrical shape have two ends with perforations to enable airflow therethrough.

Any herb may be used. Preferably the herb is non-psychoactive. More preferably, the herb is hemp.

Hemp is a common name for plants in the family of Cannabaceae, and the genus Cannabis. Hemp is a botanical class of Cannabis sativa cultivars that are specifically grown, hybridized and presented for industrial use. Industrial items made from hemp include paper, rope, textiles, clothing, biodegradable plastics, and nutrient dense food. Ideally the leaves, and not the flower of the hemp is used to make the shisha, which can be packed into a pod.

Hemp can be grown to mimic the smells, flavors and textures of marijuana, but without the psychoactive tetrahydrocannabinol component commonly associated with marijuana. Marijuana is simply a version of Cannabis sativa cultivars having a recognizable tetrahydrocannabinol (THC) content. For example, marijuana may have 1-25% THC content on a dry weight to weight (w:w) basis. Hemp is defined herein has a Cannabis sativa cultivar having less than 0.3% THC on a dry weight basis (i.e. on a weight to weight (w:w) basis).

In one embodiment of the invention dried hemp flower and leaf is used as herbage. The hemp is sorted to remove undesirable stems and foreign objects. The hemp is rinsed by boiling in water or soaked in an ethanol bath. This rinsing can be repeated several times to yield rinsed hemp. Both water soluble and ethanol soluble components rinsed from can be further separated so that undesirable waxes, chlorophyll, and contaminants can be removed and various volatile terpenes, flavonoids and flavor components can be retainer and added back to a liquid base having glycerin, honey and pectin.

In one embodiment, a pre-rinse by using ½ cup baking soda (i.e. sodium bicarbonate) in cold or warm water. High pH is a food safe rinse. This rinses many microbes away from the hemp, and renders some microbes inert due to the pH. Thus a sodium bicarbonate rinse preserves the product. This rinses soap, dirt, biological residue and and chemical residue such as pesticides. This is done prior to boiling and reducing cellular wall materials. This removes many known chemical residues prior to heating and removal of chlorophyll, which can encapsulate chemical residues.

The hemp is then dried after rinsing to nearly 0% to 12% humidity. This can be accomplished by utilizing a pressure vessel at low pressures, and heating for an extended period in a dehydrator. 453 g of moist hemp herbage can be dried to 90 g. In various embodiments the dryness can vary up to 12% moisture content. Dried hemp thus has a less than 12% moisture content.

The fully dried hemp is then rehydrated with pure water. 100 g of dried hemp material should receive 200 g of pure water to achieve a 200% hydration on a weight to weight basis. In one embodiment, the pure water is then calcified to regulate pH. In one embodiment 170 ppm of calcium is added to the purified water. In one embodiment, the re-hydrated hemp cellulose has a pH of between 6.5-8.0. In another embodiment, the re-hydrated has a pH that is greater than 7.0, and preferably 7.5.

This pH level achieves shelf stability, including maintaining the emulsification of the re-hydrated hemp and various oils that might be contained therein or added via a liquid base.

While calcium is used herein to regulate pH, it can be appreciated that other food-grade ingredients can be utilized to maintain the desired pH. Preferably all ingredients are food-grade so that the product is safely edible. In an alternate embodiment, the product is packaged in a tobacco tin and marketed to be chewed like chewing tobacco.

### Liquid Base

A liquid base is added to the cleaned, dried and rehydrated hemp. In one embodiment, the liquid base includes a combination of vegetable glycerin, honey, pectin, and various components isolated from the step of rinsing. Such components include hemp terpenes, hemp flavonoids, and volatile aromatic components. Additional flavors and scent components may be further added to the liquid base. The glycerin, the honey and the pectin cooperate to stabilize the rehydrated hemp base. The liquid base is prepared at ambient pressure at a temperature of between 142-160° F.

The liquid base is mixed with the re-hydrated hemp cellulose to reduce the moisture content on a percentage basis and so that the hemp can smolder for long periods as needed when the hemp is smoked in a shisha device, and to achieve a uniform flavor. In a preferred embodiment, the liquid base and rehydrated hemp cellulose is hand or machine stirred until V2 of the water weight has evaporated. Preferably the weight is continuously measured so that the stirring stops when the ideal water weight is reached (i.e. the ideal moisture content).

In another embodiment, the temperature of the mixture of the liquid base and the re-hydrated hemp cellulose is maintained at 200° F.-350° F. to improve the rate of evaporation. Preferably the temperature is closer to 200° F. The mixture is stirred and weighed periodically to assure desired moisture content. For example, the mixture is stirred and weighed every 30 minutes at a temperature of between 142° F. to 160° F. In this embodiment, any components that could be affected by temperatures above 200° F.-350° F. are added later, after the mixture cools to less than 120° F.

The mixture cools to less than 200° F., or preferably between 120° F. and ambient temperature after the re-hydrated hemp cellulose has its moisture content reduced appropriately. This yields a hemp base product that can be fortified with various flavors, aromatic compounds, or nutraceuticals that are stable at 120° F.

For example, the hemp base can be fortified by adding any of various non-hemp herbs, such as catnip, rose petals, lemon balm, or other herbs. The hemp base can also be fortified by adding cannabinoids. The hemp base can be packaged and sold, or modified or fortified with flavors by a retail customer, or end user. Preferably, the temperature is raised to above ambient and less than 200° F. to enable optima mixing of the fortifying components.

In a preferred embodiment, additional flavonoids, terpenoids, various cannabinoids and volatile aromatic components are added and mixed with the base hemp product. In one embodiment, these additives are sprayed when warm. In another embodiment these are mechanically mixed. Preferably the hemp base product includes a combination of vegetable glycerin, honey, pectin, as well as hemp cellulose. The various desirable components isolated from the step of rinsing may be selectively added to optimize flavor, and improve shelf life. Such components include hemp terpenes, hemp flavonoids, and volatile aromatic components. Additional flavors and scent components may be further added to the liquid base.

The product is packaged in a sealed container. No refrigeration necessary.

A method of making a smokable and edible material usable in a shisha device, or water pipe. The method includes the following steps: Providing fresh hemp leaf material and removing any non-green and foreign materials, and stems. The next step is shredding the hemp leaf material into pieces having a maximum length of 1-2″. The shredded hemp leaf material is rinsed. Rinsing the shredded hemp leave material in 3-6 washes of warm water (up to 212° F.) and 1-3 ethanol baths. The last wash is a water wash that removes any residual ethanol. The step of rinsing removes chlorophyll, undesired waxes, contaminants including heavy metals that may have been uptaken by the hemp plant during growth, yielding rinsed hemp; Drying the rinsed hemp to create dried hemp that is storable without degradation or microbial contamination; Storing the dried hemp; Processing the stored hemp by rehydrating the stored hemp to rehydrated hemp using pure water with calcium to create rehydrated hemp having a pH of greater than 7.0 to improve shelf life; Preparing a liquid base composed essentially of vegetable glycerin, pectin and honey; adding aromatic components and flavors selected from the group consisting of flavonoids, hemp terpenoids, cannabinoids, and combinations thereof to the liquid base. Next, mixing the liquid base with the rehydrated hemp at a warm temperature, and dehydrating at a temperature of preferably 200° F. The mixed materials are stirred and weighted frequently until the weight of the mixture indicates an ideal moisture content. This creates a mixed product. Adding aromatic components and flavors selected from the group consisting of flavonoids, hemp terpenoids, cannabinoids, and combinations thereof to the mixed product to create a final product. The step of adding can be accomplished at a customer location, or by an end user to enable intermediate products, such as the mixed product. The final step includes packaging the product in an air tight container, whether the final product is simply an unflavored mixed product or the final product.

An optimal method for making shisha from hemp according to the present invention includes: providing fresh hemp leaf material without crystallized cannabinoids, shredding the hemp leaf material into pieces having a maximum length of ½-2″ and preferably 1-2″ in length and a width of between 0.25 and 1″ so that when shisha is made, it has an appropriate structure to enable air flow through the shredded hemp leaf material, removing chlorophyll from the the shredded hemp leaf material by a method selected from the group consisting of aqueous rinse, acetone rinse, peroxide rinse, sun bleaching, ultraviolet curing and combinations thereof, drying shredded hemp leaf material by pressing and fluffing the hemp leaf material, placing the hemp leaf material on screens, flowing hot air over the hemp leaf material at between 180 and 220 degrees Fahrenheit, mixing a liquid base with the dried hemp leaf material, and the liquid base including material from the group consisting of vegetable glycerin pectin, honey, fructose, molasses, citric acid, flavonoids, hemp terpenoids, hemp-derived cannabinoids, and combinations thereof.

The dried hemp leaf material is one product of the present invention and this can be packaged in an airtight container to ship to customers. This can include a plastic sealed container or plastic shrink wrap, or air-tight bag. The shelf life of the dried hemp leaf material is quite long, exceeding a year. This product can be shipped to customers.

Customers are able to add a liquid base material to the dried hemp leaf material, make custom flavors and to rehydrate the hemp leaf material. Rehydrating the hemp leaf material by a distributor or brand owner yields a very fresh product with improved shelf life as measured by the date of rehydration.

The liquid base material preferably includes a combination of at least two or more hemp derived terpenes, or terpenoids.

### Terpenes in the Liquid Base

Hemp (Cannabis sativa) is known to produce a variety of terpenes, which are aromatic compounds found in many plants, including cannabis. These terpenes contribute to the distinct flavors and aromas of different hemp strains and may also have various biological functions. The invention includes the terpenes and aromatic compounds mentioned herein and others. While research on hemp terpenes is ongoing, here are some of the commonly found hemp terpenes and their potential biological functions:

Myrcene: Myrcene is one of the most abundant terpenes in hemp. It is believed to have sedative effects and may contribute to the ‘couch-lock’ feeling associated with some indica strains. Myrcene may also have anti-inflammatory and analgesic (pain-relieving) properties.

Limonene: Limonene is known for its citrusy aroma and is thought to have mood-enhancing and stress-relieving properties. It may also have antibacterial and antifungal effects.

Pinene: There are two types of pinene: alpha-pinene and beta-pinene. Pinene is responsible for the pine-like scent in some hemp strains. It is believed to have anti-inflammatory properties and may help improve airflow to the lungs. Some research suggests it may also have cognitive-enhancing effects.

Caryophyllene: Caryophyllene has a spicy, peppery aroma and is known for its potential anti-inflammatory and analgesic properties. It's unique among terpenes because it can also activate cannabinoid receptors, specifically the CB2 receptor, like a cannabinoid.

Linalool: Linalool has a floral, lavender-like scent and is often associated with relaxation and stress relief. It may have sedative properties and could potentially help with anxiety and sleep.

Humulene: Humulene has an earthy, woody aroma and may have anti-inflammatory and appetite-suppressing effect
It is also found in hops, which gives beer its distinctive flavor.

Terpinolene: Terpinolene has a complex aroma with floral, piney, and herbal notes. It is believed to have sedative properties and may also act as an antioxidant.

Ocimene: Ocimene has a sweet, herbal, and woody aroma and is thought to have antifungal and anti-inflammatory properties. It may also contribute to the overall scent profile of hemp strains.

Bisabolol: Bisabolol has a sweet, floral aroma and is known for its potential anti-inflammatory, analgesic, and skin-soothing properties. It is commonly found in skincare products.

Phytol: Phytol is derived from chlorophyll and may have antioxidant properties. It is often considered a degradation product of other terpenes.

It's important to note that the effects and functions of terpenes can vary depending on their concentration in a specific hemp strain, as well as their interactions with other compounds, including cannabinoids like THC and CBD. While these potential biological functions are based on existing research, the field of terpene research is continually evolving, and more studies are needed to fully understand their mechanisms and effects. Additionally, the specific terpene profile of a hemp strain can vary widely, leading to different sensory and potential therapeutic experiences.

### Cannabinoids in the Liquid Base

Cannabidiol (CBD) is one of the many naturally occurring compounds found in the Cannabis sativa plant. It is part of a group of compounds known as cannabinoids, which interact with the endocannabinoid system (ECS) in the human body. CBD is of particular interest because it does not produce the psychoactive effects commonly associated with another well-known cannabinoid, tetrahydrocannabinol (THC). Instead, CBD is considered non-intoxicating and is being studied for its potential therapeutic benefits.

There are several isomers of CBD, with the most common being:
CBD (Cannabidiol): This is the most prevalent and well-known isomer of CBD. It is the compound often referred to simply as “CBD.” It has a wide range of potential therapeutic effects and is the subject of numerous scientific studies.
CBDV (Cannabidivarin): CBDV is similar in structure to CBD but has a slightly different chemical arrangement. It is found in smaller quantities in the cannabis plant compared to CBD. Some research suggests that CBDV may have potential therapeutic applications, such as in the treatment of epilepsy and other neurological conditions.
CBDA (Cannabidiolic Acid): CBDA is the acidic precursor to CBD, and it is found in raw, unprocessed hemp and cannabis plants. When exposed to heat or prolonged storage, CBDA can decarboxylate and convert into CBD. Some research suggests that CBDA may have anti-inflammatory properties.

The functions of these CBD isomers in vivo (in the living organism) are still the subject of ongoing research, but here are some potential functions and effects of CBD in the body:

Interactions with the Endocannabinoid System (ECS): CBD interacts with the ECS, which plays a crucial role in regulating various physiological processes, including mood, pain perception, immune function, and more. CBD is thought to modulate the ECS without directly binding to cannabinoid receptors like THC does.

Anti-Inflammatory Properties: CBD has been studied for its potential anti-inflammatory effects, which could make it useful in managing conditions associated with inflammation, such as arthritis or inflammatory bowel diseases.

Pain Relief: Some research suggests that CBD may have analgesic (pain-relieving) properties, making it a potential option for managing chronic pain.

Anti-Anxiety and Antidepressant Effects: CBD has been investigated for its potential to reduce anxiety and depression symptoms. It may interact with serotonin receptors and influence the release of neurotransmitters related to mood regulation.

Neuroprotective Effects: CBD has shown promise in protecting nerve cells and potentially slowing down the progression of neurodegenerative diseases like Alzheimer's and Parkinson's disease.

Anti-Seizure Activity: Epidiolex, a CBD-based pharmaceutical, has been approved by the FDA for the treatment of certain forms of epilepsy, such as Dravet syndrome and Lennox-Gastaut syndrome.

Anti-Nausea and Vomiting: CBD may help reduce nausea and vomiting, particularly in individuals undergoing chemotherapy or experiencing motion sickness.

It's important to note that research into the specific functions and effects of CBD and its isomers is ongoing, and new findings continue to emerge. Additionally, the effectiveness and safety of CBD products can vary widely, so it's crucial to consult with a healthcare professional before using CBD for any specific medical condition or symptom.

The present invention can be optimized for any of the above mentioned desirable effects for particular individuals. Thus, having a liquid base formulated to have particular terpenes and cannabinoids is possible.

The synergistic effects of cannabinoids and terpenes, often referred to as the “entourage effect,” describe how these compounds in the cannabis plant can work together to produce a more significant and nuanced impact on the body and mind than when each compound is isolated. This phenomenon suggests that the combination of multiple cannabinoids and terpenes can enhance and modulate each other's effects, leading to a more holistic and beneficial experience.

Here's how the entourage effect works and why it's important:

Enhanced Therapeutic Efficacy: When multiple cannabinoids, such as CBD and THC, are present together, they can amplify each other's therapeutic benefits. For example, CBD can mitigate some of the psychoactive effects of THC, making the combination more tolerable for medical purposes while still retaining its therapeutic potential.

Modulation of Cannabinoid Receptors: Cannabinoids interact with specific receptors in the endocannabinoid system (ECS). Terpenes may influence how cannabinoids bind to these receptors. Some terpenes can enhance the binding affinity of cannabinoids to receptors, potentially increasing their effectiveness.

Diverse Pharmacological Effects: Different cannabinoids and terpenes have various pharmacological properties. When combined, they can create a broader range of effects, making it possible to target multiple symptoms or conditions simultaneously. For example, a specific combination of cannabinoids and terpenes may provide pain relief, reduce anxiety, and improve sleep.

Balancing Side Effects: Some terpenes may help counteract side effects associated with cannabinoids. For instance, the sedative properties of the terpene myrcene can offset the anxiety-inducing effects of THC, promoting relaxation.

Enhanced Bioavailability: Certain terpenes may improve the absorption and bioavailability of cannabinoids in the body, making them more effective at lower doses.

Reduced Tolerance and Dependence: Combining different cannabinoids and terpenes may potentially reduce the development of tolerance to THC, allowing individuals to achieve the same therapeutic effects with lower doses and reducing the risk of dependence.

To harness the entourage effect, individuals often prefer whole-plant or full-spectrum cannabis products that contain a wide range of cannabinoids and terpenes, rather than isolated compounds. Whole-plant extracts, such as full-spectrum CBD oil or whole-plant cannabis extracts, aim to capture the synergistic interactions between these compounds.

It's important to note that the specific entourage effect can vary depending on the unique combination of cannabinoids and terpenes in a particular cannabis strain or product. Different strains will have different terpene and cannabinoid profiles, leading to varying effects and therapeutic potentials.

As the understanding of cannabis compounds and their interactions continues to evolve, researchers are exploring the entourage effect to unlock the full therapeutic potential of this plant. However, the exact mechanisms and optimal combinations of cannabinoids and terpenes are still areas of ongoing study.

### Vaporization with a Hookah Device

The invention includes vaporization of the hemp leaf material with a hookah device.

Vaporization of cannabis is generally considered to be a healthier alternative to smoking it from a health perspective. Most hookah devices enable vaporization by heating the smokable material with charcoal or with an electric heating plate. Some have a water-pipe mechanism to cool the vapor. Here are several reasons why vaporization is considered a better option:

Reduced Harmful Chemicals: When cannabis is smoked, it undergoes combustion, which generates high temperatures and produces a variety of harmful chemicals, including carcinogens and tar. In contrast, vaporization heats the cannabis to a lower temperature, typically below the point of combustion, which means that fewer harmful byproducts are produced. This reduces the exposure to potentially harmful compounds.

Minimized Respiratory Irritation: Smoking cannabis can irritate the respiratory system due to the inhalation of hot smoke and irritants. Vaporization produces a smoother and cooler vapor that is less likely to cause irritation, coughing, or other respiratory discomfort. This can be particularly beneficial for individuals with sensitive lungs or respiratory conditions.

Preservation of Terpenes and Cannabinoids: Vaporization allows for the release of cannabinoids and terpenes from the cannabis plant without destroying them through combustion. These compounds are responsible for the therapeutic and flavor profiles of cannabis strains. By preserving these compounds, vaporization provides a more comprehensive and flavorful experience.

Controlled Temperature: Vaporizers allow users to control the temperature at which cannabis is vaporized. Different cannabinoids and terpenes have different vaporization temperatures, so users can tailor their experience to their preferences and desired effects. This level of control is not possible with smoking.

Reduced Odor: Vaporization typically produces less odor compared to smoking, making it a more discreet option for those who want to consume cannabis without drawing attention or causing strong odors in their surroundings.

Lower Risk of Secondhand Smoke: Smoking cannabis can expose others to harmful secondhand smoke, similar to tobacco smoking. Vaporization produces vapor, which is less likely to pose a risk to bystanders. This can be especially important in settings where exposure to smoke is a concern, such as in shared living spaces or public places.

Conservation of Cannabis: Vaporization tends to be more efficient in terms of cannabis consumption. It extracts cannabinoids and terpenes more effectively, meaning that less cannabis is needed to achieve the desired effects compared to smoking.

Less Carbon Monoxide: Smoking cannabis releases carbon monoxide, which can interfere with the body's ability to carry oxygen to tissues. Vaporization produces significantly less carbon monoxide, reducing the risk of oxygen deprivation.

It's important to note that while vaporization is generally considered a safer method of consuming cannabis compared to smoking, it is not completely risk-free. Some risks associated with cannabis use, such as impaired cognitive function, potential for addiction, and negative effects on mental health, are not eliminated by vaporization. Additionally, the quality of vaporization devices and the temperature used can influence the safety and effectiveness of the method.

While the present invention is described by way of examples above. The invention improves shelf life by having a dual product, i.e. dried hemp leaf material and a rehydrated material after mixing the dried hemp leaf material with a liquid base, the present invention can have the many health benefits of hemp, and the ability for a customer to custom formulate products that are saleable to end users. This is due to the matrix structure of the shredded hemp leaf material that enables air flow through the shredded hemp material, even after the liquid base is mixed with the shredded hemp material.

The true scope of the invention is set forth in the appended claims.

We claim:

1. A method for making shisha from hemp, comprising:
a. providing fresh hemp leaf material without crystallized cannabinoids;
b. shredding the hemp leaf material into pieces having a maximum length of ½-2″ and preferably 1-2″ in length and a width of between 0.25 and 1″ so that when shisha is made, it has an appropriate structure to enable air flow through the shredded hemp leaf material;
c. removing chlorophyll from the the shredded hemp leaf material by a method selected from the group consisting of aqueous rinse, acetone rinse, peroxide rinse, sun bleaching, ultraviolet curing and combinations thereof;
d. drying shredded hemp leaf material by pressing and fluffing the hemp leaf material, placing the hemp leaf material on screens, flowing hot air over the hemp leaf material at between 180 and 220 degrees Fahrenheit.

2. The method as set forth in claim 1 further comprising:
packaging and storing the dried and shredded hemp leaf material.

3. The method as set forth in claim 1 further comprising:
packaging and storing the dried and shredded hemp leaf material and then mixing a liquid base with the dried hemp leaf material.

4. The method as set forth in claim 3, wherein the liquid base includes material from the group consisting of vegetable glycerin pectin, honey, fructose, molasses, citric acid, flavonoids, hemp terpenoids, hemp-derived cannabinoids, and combinations thereof.

5. The method as set forth in claim 4, wherein the liquid base includes cannabidiol derived from hemp.

6. The method as set forth in claim 4, wherein the liquid base includes at least one terpene derived from hemp.
